(19) United States
(12) Patent Application Publication
(10) Pub. No.: US 2026/0020901 A1
(43) Pub. Date: Jan. 22, 2026

(54) TREATMENT TOOL FOR ENDOSCOPE

(71) Applicants: OLYMPUS MEDICAL SYSTEMS CORP., Tokyo (JP); Haruhiro INOUE, Yokohama-shi (JP)

(72) Inventors: Junichi KOGISO, Hachioji-shi (JP); Chikia MIYAJIMA, Hachioji-shi (JP); Haruhiro INOUE, Yokohama-shi (JP)

(21) Appl. No.: 19/340,230

(22) Filed: Sep. 25, 2025

Related U.S. Application Data
(63) Continuation of application No. PCT/JP2024/000935, filed on Jan. 16, 2024.
(60) Provisional application No. 63/492,883, filed on Mar. 29, 2023.

(51) Int. Cl. A61B 18/14; A61B 18/00

(52) U.S. Cl. CPC A61B 18/1482; A61B 2018/00077; A61B 2018/00083; A61B 2018/00101; A61B 2018/00482; A61B 2018/00577; A61B 2018/00607; A61B 2018/00982; A61B 2018/1475

(57) ABSTRACT

A treatment tool for an endoscope includes a sheath having insulation properties can include a wire provided inside the sheath so as to be advanceable and retractable and to be electrifiable with a high-frequency current. An electrode can be connected to a distal end of the wire. In a state in which a distal end of the electrode is positioned inside the sheath, hemorrhage is controllable by pressing a distal end of the sheath against a tissue and discharging electricity while maintaining a distance from the tissue to the electrode.

### CLAIM OF PRIORITY

This application is a continuation application based on International Patent Application No. PCT/JP2024/000935 filed on Jan. 16, 2024, whose priority is claimed on U.S. Provisional Application No. 63/492,883, filed Mar. 29, 2023. Both of the content of the PCT International Application and the Japanese Application are incorporated herein by reference.

### BACKGROUND

A treatment tool for an endoscope such as high-frequency knife can be used in endoscopic therapy such as endoscopic submucosal dissection (ESD). In such procedures, a practitioner such as a surgeon performs incision in a tissue of a living body and coagulation hemostasis using the treatment tool.

A hemostatic method for controlling hemorrhage can include discharging electricity in a non-contact state in which a distal end of a high-frequency knife is kept away from a hemorrhage site in a tissue of a living body to cause the hemorrhage site in the tissue of the living body to thermally deform.

### SUMMARY

A treatment tool for an endoscope according to a first aspect of the present disclosure can include a sheath having insulation properties, a wire provided inside the sheath so as to be advanceable and retractable and to be electrifiable with a high-frequency current, and an electrode connected to a distal end of the wire. In a state in which a distal end of the electrode is positioned inside the sheath, hemorrhage can be controllable by pressing a distal end of the sheath against a tissue and discharging electricity while maintaining a distance from the tissue to the electrode.

### BRIEF DESCRIPTION OF THE DRAWINGS

FIG. 1 is an example of a general view of an endoscopic treatment system according to a first embodiment.

FIG. 2 is an example of a general view showing a treatment tool of an endoscopic treatment system.

FIG. 3 is an example of a perspective view of a distal end part of a treatment tool.

FIG. 4 is an example of a cross-sectional view of the distal end part of a treatment tool.

FIG. 5 is an example of a cross-sectional view of the distal end part of a treatment tool.

FIG. 6 is an example of a front view of the distal end part of a treatment tool.

FIG. 7 is an example of a front view of a flange of a treatment tool.

FIG. 8 is an example of a front view of a flange of a treatment tool.

FIG. 9 is an example of a view showing a hemostasis step using a treatment tool.

FIG. 10 is an example of a view showing a hard part of a treatment tool.

FIG. 11 is an example of a distal end tubular part of a treatment tool.

FIG. 12 is an example of a distal end tubular part of a treatment tool.

FIG. 13 is an example of a distal end tubular part of a treatment tool.

FIG. 14 is an example of a distal end tubular part of a treatment tool.

FIG. 15 is an example of a distal end tubular part of a treatment tool.

FIG. 16 is an example of a cross-sectional view of a distal end part of a treatment tool according to a second embodiment.

FIG. 17 is an example of a rod of a treatment tool.

FIG. 18 is an example of a rod of a treatment tool.

FIG. 19 is an example of a rod of a treatment tool.

FIG. 20 is an example of a perspective view of a distal end part of a treatment tool according to a third embodiment.

FIG. 21 is an example of a cross-sectional view of a distal end part of a treatment tool.

FIG. 22 is an example of a perspective view of a cap of a treatment tool.

FIG. 23 is an example of a perspective of a cap of a treatment tool.

FIG. 24 is an example of a perspective view showing a treatment tool according to a fourth embodiment.

### DETAILED DESCRIPTION

### First Embodiment

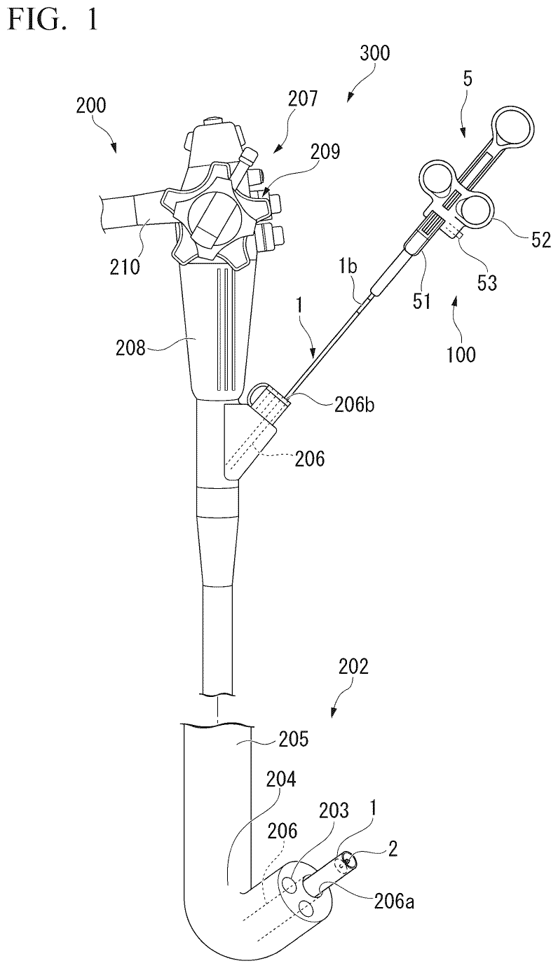

An endoscopic treatment system 300 according to a first embodiment of the present disclosure will be described with reference to FIGS. 1 to 15. FIG. 1 is an example of a general view of the endoscopic treatment system 300 according to the present embodiment.

### [Endoscopic Treatment System 300]

As shown in FIG. 1, the endoscopic treatment system 300 can include an endoscope 200 and a treatment tool 100. The treatment tool 100 can be used by being inserted into the endoscope 200.

### [Endoscope 200]

The endoscope 200 can be flexible endoscope, which can include an insertion part 202 configured to be inserted into a human body from its distal end, and a manipulation part 207 attached to a proximal end of the insertion part 202.

The insertion part 202 can include an image capturing part 203, a bending part 204, and a flexible part 205. The image capturing part 203, the bending part 204, and the flexible part 205 can each arranged in this order from the distal end of the insertion part 202. A channel 206 for inserting the treatment tool 100 can be provided inside the insertion part 202. A distal end opening part 206a of the channel 206 can be provided at the distal end of the insertion part 202.

For example, the image capturing part 203 can include an image capturing element such as a Charged-Coupled Device (CCD), a Complementary Metal-Oxide Semiconductor (CMOS), or the like, and can capture an image of a part to be treated. The image capturing part 203 can capture an image of a rod 2 of the treatment tool 100 in a state in which the treatment tool 100 protrudes from the distal end opening part 206a of the channel 206.

The bending part 204 can bend in response to a manipulation of the manipulation part 207 by a manipulator. The flexible part 205 can be or include a tubular part having flexibility.

The manipulation part 207 can be connected to the flexible part 205. The manipulation part 207 can have or include a grip 208, an input part 209, a proximal end opening part 206b of the channel 206, and a universal cord 210. The grip 208 can be a part grasped by a manipulator. The input part 209 can receive a manipulation input for a bending motion of the bending part 204. The universal cord 210 can output, transmit, or the like an image captured by the image capturing part 203 (e.g., a signal representing the image captured by the image capturing part 203) to the outside. The universal cord 210 can be connected to a display device such as a liquid crystal display (LCD) via an image processing device including a processor, processing circuitry, or the like.

[Treatment tool 100]

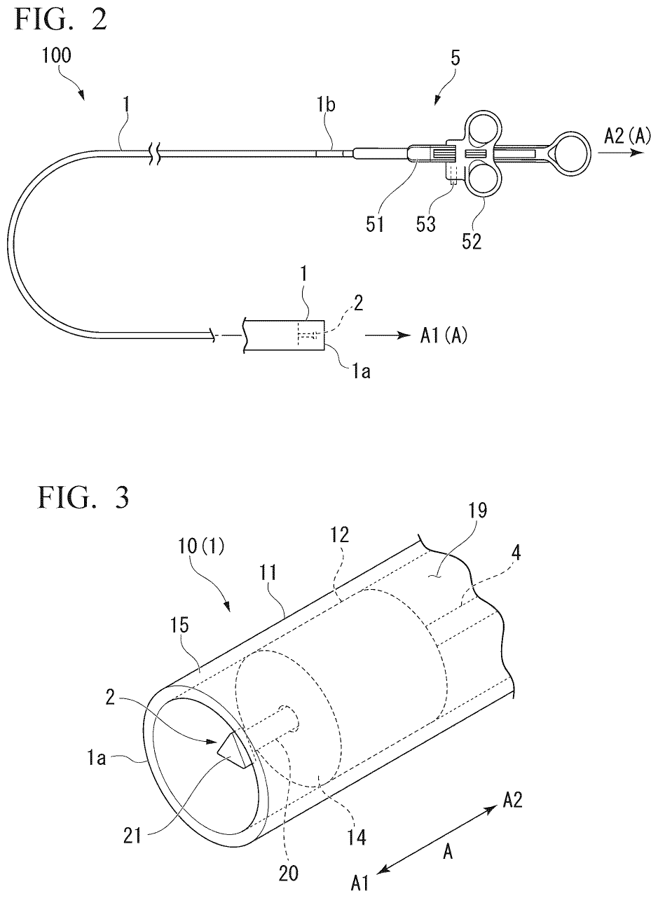

FIG. 2 is an example of a general view showing the treatment tool 100. The treatment tool (treatment tool for an endoscope) 100 can be or include an ESD knife capable of performing incision and coagulation hemostasis. The treatment tool 100 can include a sheath 1, the rod 2, a manipulation wire 4 (e.g., as illustrated in FIG. 3), and a manipulation part 5. In the following description, in a longitudinal direction A of the treatment tool 100, a side to be inserted into the body of a patient will be referred to as “a distal end side (distal side) A1”, and the manipulation part 5 side will be referred to as “a proximal end side (proximal side) A2”.

The sheath 1 can be or include a long tubular member extending from a distal end 1a to a proximal end 1b. The sheath 1 can have or include an outer diameter allowing it to be inserted into the channel 206 of the endoscope 200 and can be advanceable and retractable in the channel 206. As shown in FIG. 1, in a state in which the sheath 1 is inserted into the channel 206, the distal end 1a of the sheath 1 can project and retract from the distal end opening part 206a of the channel 206.

FIG. 3 is an example of a perspective view of a distal end part of the treatment tool 100. The sheath 1 can have or include a tube 11 extending in the longitudinal direction A, and a hard part 12 (e.g., a rigid part or portion) attached to a distal end part 10 of the sheath 1.

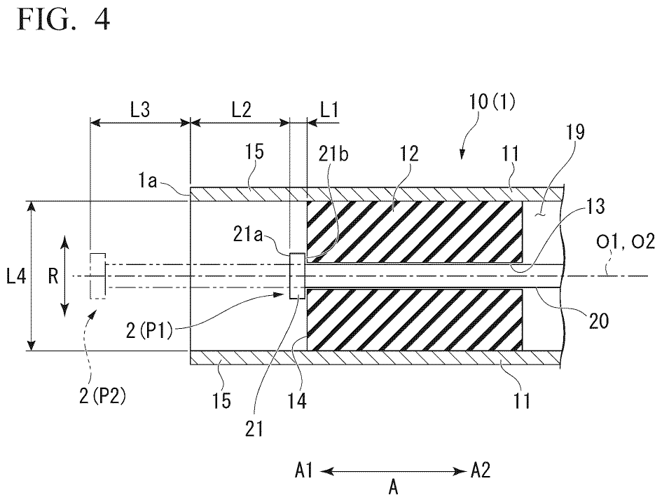

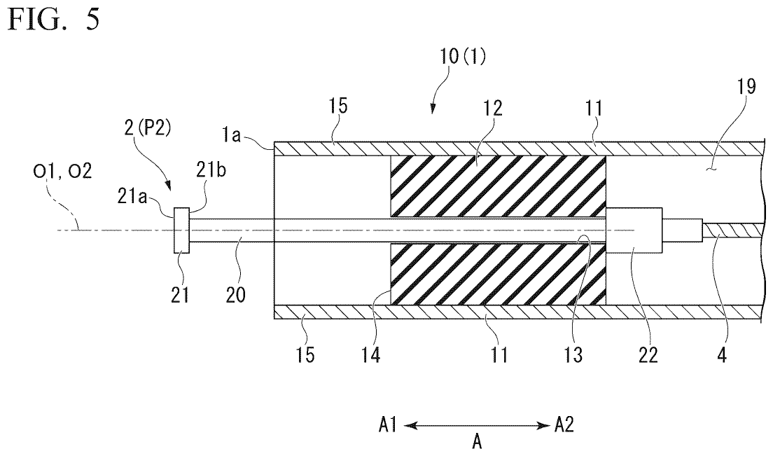

FIGS. 4 and 5 are examples of cross-sectional views of the distal end part 10 of the treatment tool 100. The tube 11 can be or include a long tubular member having flexibility and electrical insulation properties. For example, the tube 11 can be formed of a resin or a similar material.

The hard part (distal end tip) 12 can be provided so as to occlude an internal space 19 of the tube 11. The hard part 12 can have or include a penetration hole 13 penetrating it in the longitudinal direction A. A rod main body 20 of the rod 2 can be inserted into the penetration hole 13. The penetration hole 13 of the distal end tip 12 can communicate with the internal space 19 (e.g., a conduit, lumen, or the like) of the tube 11.

The hard part 12 can be attached to the proximal end side A2 from the distal end 1a of the sheath 1 in the distal end part 10 of the sheath 1. For this reason, in the distal end part 10 of the sheath 1, the distal end part of the tube 11 can be positioned on the distal end side A1 from the hard part 12. The distal end part of the tube 11 positioned on the distal end side A1 from the hard part 12 in the distal end part 10 of the sheath 1 will be referred to as “a distal end tubular part 15”. In the present embodiment, the distal end tubular part 15 is a portion of the tube 11. For example, the distal end tubular part 15 can be formed to include at least one of a ceramic, zirconia, polytetrafluoroethylene (PTFE), and polyetheretherketone (PEEK).

In an example, the distal end tubular part 15 can have a transparency sufficient to allow the rod 2 to be visually recognized from the outward side of the sheath 1. In such an example, the distal end tubular part 15 can be formed of a transparent material such as PTFE. Making the distal end tubular part transparent or semi-transparent can help, aid, assist, or the like a practitioner to check the position of the rod 2 from an image captured by the image capturing part 203 of the endoscope 200.

In an example, rod 2 can be a metal member (e.g., an electrode) having a shape of a round bar (e.g., a cylindrical or substantially cylindrical shape). In this context, the “shape of a round bar” is not limited to a strict cylindrical shape. However, it need only be formed to have almost a shape of a round bar, and design errors or modifications are allowed. In an example, the rod 2 can be formed using a raw material such as stainless steel. The rod 2 can be electrically conductive and can be electrified with a high-frequency current. The rod 2 can have or include a rod main body 20, a flange 21, and a large diameter part 22.

The rod 2 can be inserted through the penetration hole 13 of the hard part 12 of the sheath 1 in the longitudinal direction A so as to be advanceable and retractable. The rod 2 can be advanceable and retractable from a first position P1 where the distal end of the rod 2 can be positioned on the distal side A1 from the hard part 12 inside the sheath 1 to a second position P2 where the distal end of the rod 2 can be positioned on the distal side A1 from the distal end 1a of the sheath 1. A center axis O2 of the rod 2 in the longitudinal direction A can coincide, align, line up with, or the like with a center axis O1 of the sheath 1 in the longitudinal direction A. There is no need for the center axis O2 to perfectly or exactly coincide with the center axis O1, and modifications or errors (e.g., errors due to an influence of assembly errors or the like) are allowed.

The rod main body 20 (electrode main body) can be or include a metal member having a shape of a round bar. The manipulation wire 4 can be attached to the proximal end of the rod main body 20. A high-frequency (e.g. Radio Frequency (RF)) current can be supplied to the rod main body 20 from the manipulation wire 4 connected to the manipulation part 5. When a high-frequency current is supplied to the rod 2 from the manipulation wire 4, the rod main body 20 and the flange 21 can function as a monopolar electrode outputting a high-frequency current to a tissue of a living body.

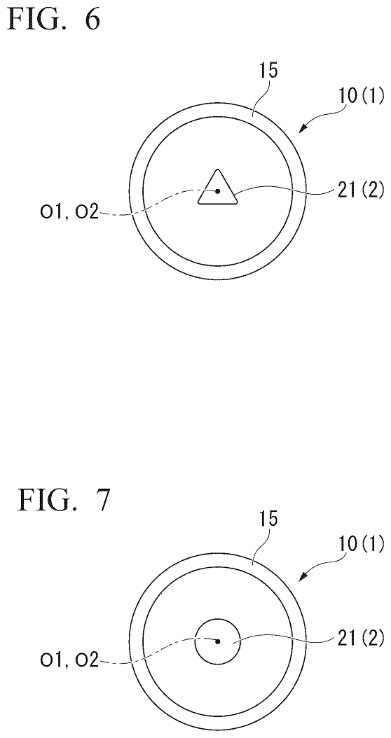

FIG. 6 is an example of a front view of the distal end part of the treatment tool 100. The flange 21 (e.g., an enlarged diameter part) can be a conductive member having a plate shape (or a substantially plate shape) provided at the distal end of the rod main body 20. In a front view in a direction extending in the longitudinal direction A, the flange 21 can be formed to have a triangular shape. The length of the flange 21 in a radial direction R perpendicular to the longitudinal direction A can be (can be made or formed) longer than the length of the rod main body 20 in the radial direction R. As shown in FIG. 4, the flange 21 can come into contact with the hard part 12 so that the rod 2 is positionally set at the first position P1.

A proximal end surface 21b (e.g., a rear surface) having a planar shape can be formed on the proximal end side A2 of the flange 21. As shown in FIG. 4, when the rod 2 is positioned at the first position P1, the proximal end surface 21b of the flange 21 can abut a distal end part 14 of the hard part 12.

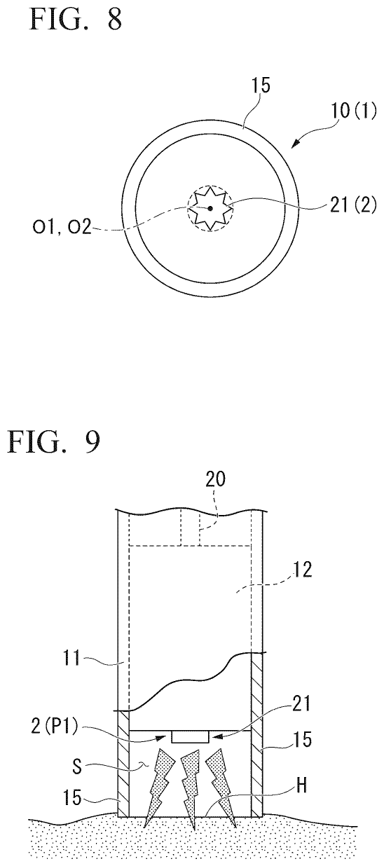

FIGS. 7 and 8 are examples of front views of modification examples of the flange 21. In a front view in a direction extending in the longitudinal direction A, the flange 21 can have a circular shape or can have a polygonal shape. From the viewpoint of outputting a high-frequency current to a tissue of a living body, the flange 21 can have a polygonal shape with many corners. Moreover, from the viewpoint of efficiently generating sparks within an intended range, the distal end tubular part 15 can be disposed concentrically with the rod 2 in a front view in the longitudinal direction A and the distal end tubular part 15 can be disposed concentrically with a virtual circle connecting apexes of the polygonal shape. In an example, a plurality of apexes in the outermost circumference of the flange 21 can be positioned on a virtual circle concentric with the rod 2.

The large diameter part 22 (e.g. a stopper) can be provided on the proximal end side A2 from the hard part 12. The large diameter part 22 can have or be formed with a larger diameter dimension (length in the radial direction R) than the rod main body 20. As shown in FIG. 5, in the rod 2, the large diameter part 22 can come into contact with the hard part 12 and can be positionally set at the second position P2.

As shown in FIG. 4, a second distance L2 from the distal end of the rod 2 positioned at the first position P1 to the distal end 1a of the sheath 1 can be longer than a first distance L1 from the distal end of the rod 2 positioned at the first position P1 to the distal end of the hard part 12.

As shown in FIG. 4, the second distance L2 from the distal end of the rod 2 positioned at the first position P1 to the distal end 1a of the sheath 1 can be substantially equivalent to a third distance L3 from the distal end of the rod 2 positioned at the second position P2 to the distal end 1a of the sheath 1. In an example, the relationship between a fourth distance L4, which is the inner diameter of the sheath 1 (distance in the radial direction R), and the second distance L2 can be 0<L2/L4≤2 when the distal end 1a of the sheath 1 is pressed against a tissue such that the tissue positioned on the outward side of the sheath 1 is unlikely to enter the inward side of the sheath 1. In another example, the relationship therebetween can be 0<L2/L4≤1.

The manipulation wire 4 can be a metal wire inserted through the internal space (conduit, lumen) 19 of the tube 11. For example, the manipulation wire 4 can be formed using a raw material such as stainless steel. The distal end of the manipulation wire 4 can be connected to the rod 2, and the proximal end of the manipulation wire 4 can be connected to the manipulation part 5.

As shown in FIGS. 1 and 2, the manipulation part 5 can have or include a manipulation part main body 51, a slider 52, and a power feeding connector 53.

The distal end part of the manipulation part main body 51 can be connected to the proximal end 1b of the sheath 1. The manipulation part main body 51 can have or include an internal space through which the manipulation wire 4 can be inserted. The manipulation wire 4 can pass through the internal space 19 of the tube 11 and the internal space of the manipulation part main body 51 and can extend to the slider 52.

The slider 52 can be attached to the manipulation part main body 51 so as to be movable in the longitudinal direction A. A proximal end part of the manipulation wire 4 can be attached to the slider 52. When a practitioner causes the slider 52 to advance and retract relative to the manipulation part main body 51, the manipulation wire 4 and the rod 2 advance and retract.

The power feeding connector 53 can be fixed to the slider 52. The power feeding connector 53 can be connected to a high-frequency power source device, and can be connected to the proximal end part of the manipulation wire 4 via a conductive wire. The power feeding connector 53 can supply a high-frequency current supplied from the high-frequency power source device to the rod 2 via the manipulation wire 4. In an example, the power feeding connector 53 can be fixed to the manipulation part main body 51 instead of the slider 52.

### [Method for Using Endoscopic Treatment System 300]

Next, a technique using the endoscopic treatment system 300 of the present embodiment (method for using the endoscopic treatment system 300) will be described. Specifically, local injection treatment, incision/ablation treatment, and hemostasis treatment for a lesion in endoscopic therapy such as endoscopic submucosal dissection (ESD) will be described.

Regarding preparation work, a practitioner (e.g., a physician, surgeon, or the like) can identify a lesion. For example, a practitioner can insert the insertion part 202 of the endoscope 200 into the digestive tract (for example, the esophagus, the stomach, the duodenum, or the large intestine) and identify a lesion while observing the image obtained by the image capturing part 203 of the endoscope.

### <Insertion Step>

The practitioner can insert the treatment tool 100 into the channel 206 and cause the distal end 1a of the sheath 1 to protrude from the distal end opening part 206a of the insertion part 202. The practitioner can cause the slider 52 of the manipulation part 5 to advance relative to the manipulation part main body 51 and causes the rod 2 to protrude.

### <Incision/Ablation Step>

The practitioner can perform an incision/ablation treatment. The practitioner can perform an incision of a mucous membrane of a lesion by causing the rod 2 to advance to the second position P2 and moving the flange 21 in a state of being electrified with a high-frequency current. In addition, the practitioner can cause the rod 2 to advance to the second position P2 and ablate the lower layer of the incised mucous membrane of the lesion while lifting up the incised mucous membrane of the lesion and exposing the lower layer of the mucous membrane in a state of being electrified with a high-frequency current.

### <Hemostasis Step>

FIG. 9 is an example of a view showing a hemostasis step using the treatment tool 100. When hemorrhage occurs during an incision or ablation treatment, the practitioner can perform hemostasis treatment. In an example, the practitioner can cause the rod 2 to retract to the first position P1. The practitioner can press the distal end 1a of the sheath 1 against a tissue of a living body so that the distal end 1a of the sheath 1 can adhere to the tissue of the living body. The practitioner can surround a hemorrhage site H in the tissue of the living body with the distal end tubular part 15. This can help allow the practitioner to maintain a non-contact state in which the distal end (flange 21) of the rod 2 is kept away from the hemorrhage site H in the tissue of the living body at an appropriate distance.

In an example, no insulating material member is interposed between the flange 21 and the sheath distal end 1a (e.g. distal end 1a of a distal tube 15,15B). In addition, no insulating material member may be present on the distal side A1 from the flange 21 in the longitudinal direction A of the rod 2. For this reason, there may be no shield between the flange 21 (electrode) and the target tissue, and the area around the electrode and the target tissue can be in a state of being covered by an electrical insulating material member. Therefore, the tissue can be cauterized in a shape along the edge of an opening at the sheath distal end 1a.

If or when the rod 2 comes into contact with the hemorrhage site H in the tissue of the living body, it can cause a rise in impedance of the tissue of the living body, carbonization, transpiration, or the like so that a sufficient hemostatic effect cannot be achieved. Meanwhile, if or when the rod 2 is excessively away (e.g., a threshold distance) from the hemorrhage site H in the tissue of the living body, electricity cannot be discharged with respect to the hemorrhage site H.

Thus, the practitioner can control hemorrhage by discharging electricity in a non-contact state in which the distal end (flange 21) of the rod 2 is kept away from the hemorrhage site H in the tissue of the living body at an appropriate distance to cause the hemorrhage site H in the tissue of the living body to thermally deform. Although the hemorrhage site H and the flange 21 are kept away at an appropriate distance, the hemorrhage site H can be positioned in a discharge space S surrounded by the distal end tubular part 15. For this reason, electricity is not discharged to the outward side of the sheath 1, and electricity can be discharged within an intended range with respect to the hemorrhage site H. As a result, the hemorrhage site H can thermally deform into a circular shape.

The practitioner can continue the motion (treatment) described above as necessary and eventually excises the lesion, thereby ending the technique of ESD.

According to the treatment tool 100 of the present embodiment, a non-contact state can be maintained in which the distal end (flange 21) of the rod 2 is kept away from the hemorrhage site H in the tissue of the living body at an appropriate distance. In addition, the hemorrhage site H can be surrounded by the distal end tubular part 15. For this reason, the treatment tool 100 can discharge electricity within an intended range with respect to the hemorrhage site H.

Since the second distance L2 and the third distance L3 are substantially equivalent to each other, the practitioner can perform incision treatment or ablation treatment by disposing the rod 2 at the second position P2, and can perform hemostasis treatment by disposing the rod 2 at the first position P1 and discharging electricity with respect to the hemorrhage site H.

Hereinabove, the first embodiment of the present disclosure has been described in detail with reference to the drawings, but specific constitutions are not limited to this embodiment, and design change and the like within a range not departing from the scope of the present disclosure are also included therein. In addition, the constituent elements shown in the embodiment and the modification example described above can be constituted in suitable combinations.

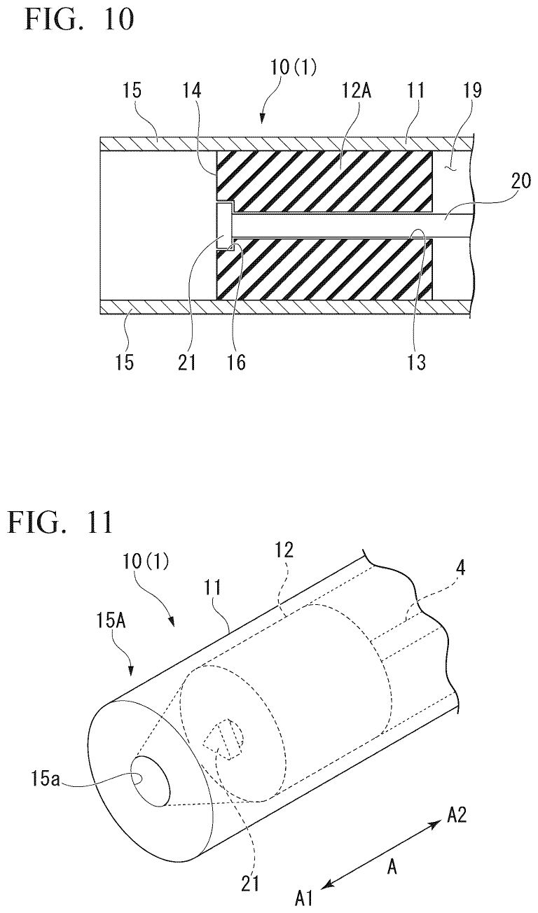

FIG. 10 is an example of a view showing a hard part 12A which is a modification example of the hard part 12. The hard part 12A can have or include a recessed part 16 capable of accommodating the flange 21 of the rod 2 on the distal end side A1 of the penetration hole 13. A practitioner may discharge electricity with respect to the hemorrhage site H in a state in which the flange 21 is accommodated in the recessed part 16.

FIG. 11 is an example of a view showing a distal end tubular part 15A which is a modification example of the distal end tubular part 15. The inner diameter of an opening 15a (opening diameter) at the distal end of the distal end tubular part 15A can be made or formed smaller than the outer diameter of the hard part 12. In addition, the inner circumferential surface of the distal end tubular part 15A can be formed to have a tapered shape such that the inner diameter decreases toward the distal end of the distal end tubular part 15A. In such an example, the discharge space S in which the distal end tubular part 15A surrounds the hemorrhage site H becomes smaller.

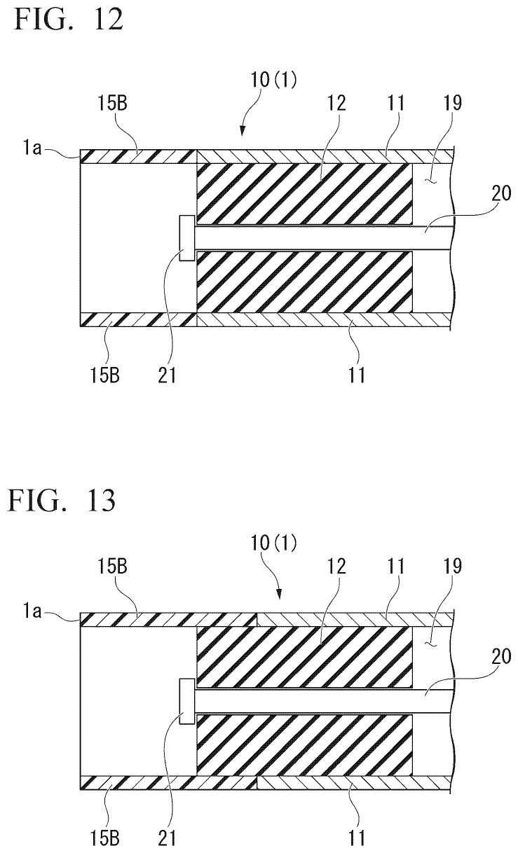

FIGS. 12 and 13 are example views showing a distal end tubular part 15B which is a modification example of the distal end tubular part 15. The distal end tubular part 15B can be a member different from the tube 11 and can be formed using a material having an excellent heat resistance compared to the tube 11. The distal end tubular part 15B need only be a member having a high heat resistance and does not necessarily have to be a different member. For example, the distal end tubular part 15B can be formed to include at least one of a ceramic, zirconia, PTFE, and PEEK. Due to the distal end tubular part 15B being provided in the distal end part 10 of the sheath 1, the heat resistance can be improved while retaining the flexibility of the sheath 1. As shown in FIG. 13, the distal end tubular part 15B and the tube 11 can be joined on the outer circumferential surface of the hard part 12. In addition to joining the end part of the distal end tubular part 15B and the end part of the tube 11, the distal end tubular part 15B and the tube 11 can be joined on the outer circumferential surface of the hard part 12 by bonding or the like, thereby making the two more firmly joined.

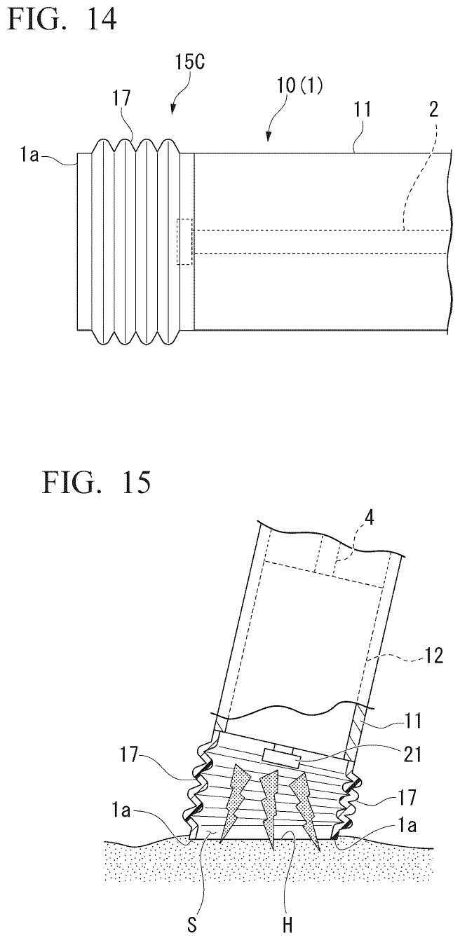

FIGS. 14 and 15 are example views showing a distal end tubular part 15C which is a modification example of the distal end tubular part 15. The distal end tubular part 15C can have or include an extension/contraction part 17 capable of extending and contracting. The extension/contraction part 17, shown in FIG. 14 as an example, can be formed to have a bellows shape. In the distal end tubular part 15C, as shown in FIG. 15, even when the distal end part 10 of the sheath 1 cannot be pressed against the hemorrhage site H in the tissue of the living body from the front, the extension/contraction part 17 can extend and contract so that the whole circumference of the distal end 1a of the sheath 1 can be brought into contact with a tissue of a living body. As a result, the degree of sealing of the discharge space S can be enhanced.

### Second Embodiment

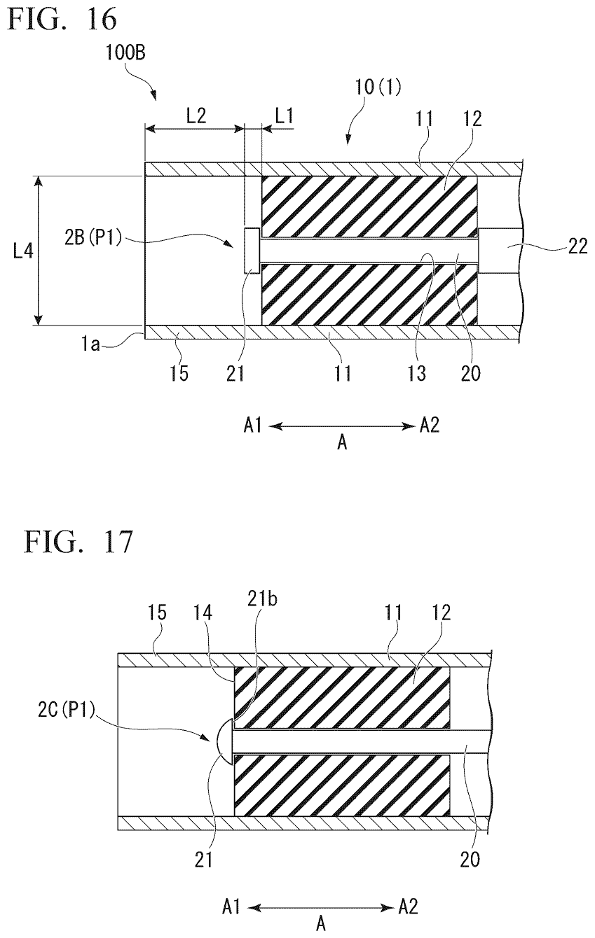

A treatment tool 100B according to a second embodiment of the present disclosure will be described with reference to FIGS. 16 to 19. In the following description, the same reference signs are applied to constituents common to those which have already been described, and duplicate description will be omitted.

FIG. 16 is an example of a cross-sectional view of the distal end part of the treatment tool 100B. The treatment tool 100B (e.g., treatment tool for an endoscope) can be a dedicated hemostatic instrument capable of performing coagulation hemostasis. The treatment tool 100B can include the sheath 1, a rod 2B, the manipulation wire 4, and the manipulation part 5. The treatment tool 100B can differ from the treatment tool 100 of the first embodiment in that the rod 2B does not advance and retract. Similarly to the treatment tool 100 of the first embodiment, the treatment tool 100B can be used by being inserted into the channel 206 of the endoscope 200.

The rod 2B can be inserted through the penetration hole 13 formed in the hard part 12 so as to be not advanceable and retractable. The rod 2B can be positionally set at the first position P1 where the distal end of the rod 2B is positioned on the distal side A1 from the hard part 12 inside the sheath 1. That is, the distal end of the rod 2B can be positionally set between the distal end of the distal end tubular part 15 and the hard part 12. Specifically, the rod 2B can be fixed at the first position P1 by sandwiching the hard part 12 between the flange (enlarged diameter part) 21 and the large diameter part 22, and the tip of the rod 2B, that is, the flange (enlarged diameter part) 21 can be constituted so as not to protrude from the distal end of the sheath 1. The expression “not advanceable and retractable” denotes that advancing and retracting are not required as a function, and errors which may occur in design or manufacturing, rattling due to clearances, and the like are included in the form of “not advanceable and retractable”.

The second distance L2 from the distal end of the rod 2B to the distal end 1a of the sheath 1 can be longer than the first distance L1 from the distal end of the rod 2B to the distal end of the hard part 12. In an example, the relationship between the fourth distance L4, which is the inner diameter of the sheath 1 (distance in the radial direction R), and the second distance L2 be 0<L2/L4≤2 when the distal end 1a of the sheath 1 is pressed against a tissue such that the tissue positioned on the outward side of the sheath 1 is unlikely to enter the inward side of the sheath 1. In another example, the relationship therebetween can be 0<L2/L4≤1.

Similarly to the treatment tool 100 of the first embodiment, a practitioner can control hemorrhage in the hemorrhage site H using the treatment tool 100B.

According to the treatment tool 100B of the present embodiment, a non-contact state can be maintained in which the distal end (flange 21) of the rod 2B is kept away from the hemorrhage site H in the tissue of the living body at an appropriate distance. In addition, the hemorrhage site H can be surrounded by the distal end tubular part 15. For this reason, the treatment tool 100B can discharge electricity within an intended range with respect to the hemorrhage site H.

Hereinabove, the second embodiment of the present disclosure has been described in detail with reference to the drawings, but specific constitutions are not limited to this embodiment, and design change and the like within a range not departing from the scope of the present disclosure are also included therein. In addition, the constituent elements shown in the embodiment and the modification example described above can be constituted in suitable combinations.

FIGS. 17 and 18 are example views showing a rod 2C which is a modification example of the rod 2B. In an example rod 2C does not have the large diameter part 22. The distal end of the rod 2C can be fixed to the hard part 12, and thus it is fixed at the first position P1. The proximal end surface 21b of the flange 21 can be disposed so as to abut the distal end part 14 of the hard part 12 or can be disposed slightly spaced apart from the distal end part 14 of the hard part 12. As in FIG. 18, the rod 2C can be fixed to the hard part 12 by hooking a plurality of projections 20a (e.g., concave and convex parts) provided around the rod main body 20 onto the hard part 12.

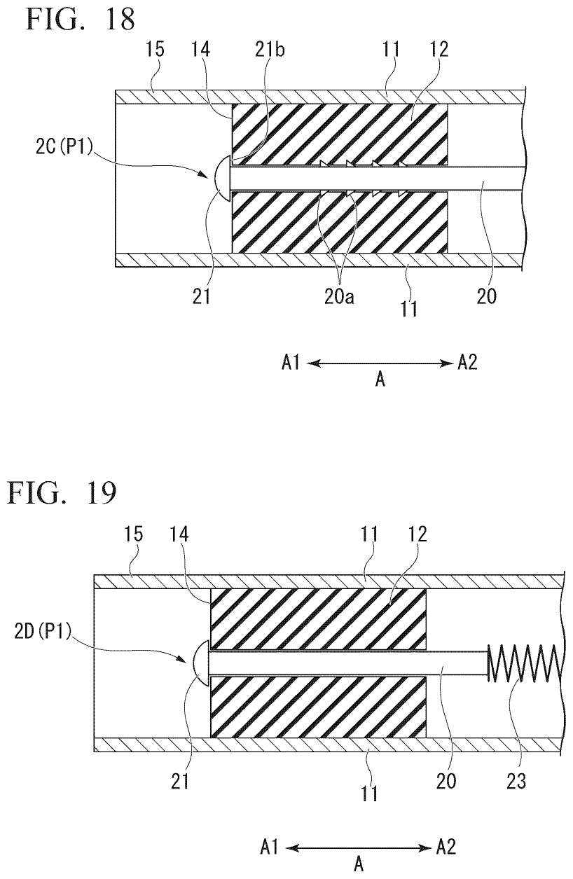

FIG. 19 is an example of a view showing a rod 2D which is a modification example of the rod 2B. The rod 2D can be positionally set at the first position P1 by being biased to the proximal end side A2 by an elastic member 23 such as a spring.

In the distal end tubular part 15 of the treatment tool 100B, as in the distal end tubular part 15A shown in FIG. 11, the inner circumferential surface can be formed to have a tapered shape in which the inner diameter decreases toward the distal end. In addition, as in the distal end tubular part 15B shown in FIGS. 12 and 13, the distal end tubular part 15 of the treatment tool 100B can be a member different from the tube 11 and can be formed using a material having an excellent heat resistance compared to the tube 11.

### Third Embodiment

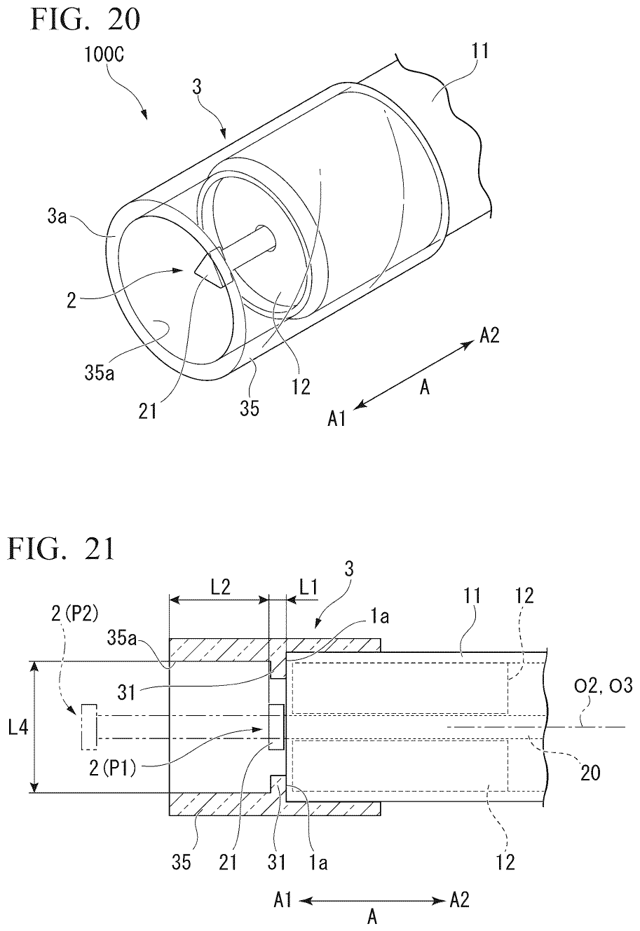

A treatment tool 100C according to a third embodiment of the present disclosure will be described with reference to FIGS. 20 to 23. In the following description, the same reference signs are applied to constituents common to those which have already been described, and duplicate description will be omitted.

FIG. 20 is an example of a perspective view of the distal end part of the treatment tool 100C. The treatment tool 100C (treatment tool for an endoscope) can be a tool in which a cap 3 (e.g., a sheath tube) can be mounted in an ESD knife capable of performing incision and coagulation hemostasis. The treatment tool 100C can include a sheath 1C, the rod 2, the manipulation wire 4, and the manipulation part 5. Similarly to the treatment tool 100 of the first embodiment, the treatment tool 100C can be used by being inserted into the channel 206 of the endoscope 200.

Similarly to the sheath 1 of the first embodiment, the sheath 1C can be a long tubular member extending from the distal end 1a to the proximal end 1b. The sheath 1 can have or include a tube 11C extending in the longitudinal direction A, and the hard part 12 can be attached to the distal end part 10 of the sheath 1. The tube 11C can differs from the tube 11 of the first embodiment such that it does not have the distal end tubular part 15 extending to the distal end side A1 of the hard part 12.

FIG. 21 is an example of a cross-sectional view of the distal end part of the treatment tool 100C. The cap 3 (sheath tube) can be a tubular member which can be attachable and detachable with respect to the distal end of the sheath 1 and can have or include electrical insulation properties. The cap 3 can have or include a step 31 on the inner circumferential surface. When the cap 3 is attached to the outer circumferential surface of the distal end of the sheath 1, the step 31 can bump into, make contact with, or the like, the distal end 1a of the sheath 1 so that the cap 3 is positionally set with respect to the sheath 1. In the present embodiment, the step 31 can be a projection protruding from the inner circumferential surface to the inward side in the radial direction R.

The rod 2 can be inserted through the penetration hole 13 of the hard part 12 of the sheath 1 in the longitudinal direction A so as to be advanceable and retractable. The rod 2 can be advanceable and retractable from the first position P1 where the distal end of the rod 2 is positioned on the distal side A1 from the hard part 12 inside the cap 3 to the second position P2 where the distal end of the rod 2 is positioned on the distal side A1 from a distal end 3a of the cap 3. The center axis O2 of the rod 2 in the longitudinal direction A can substantially coincides with (e.g., align with, or the like) a center axis O3 of the cap 3 in the longitudinal direction A.

As shown in FIG. 21, the flange 21 can come into contact with the hard part 12 so that the rod 2 can be positionally set at the first position P1. In a state in which the cap 3 is attached to the distal end of the sheath 1, the distal end of the cap 3 can be positioned on the distal side A1 from the distal end of the rod 2 when the rod 2 is positionally set at the first position P1.

The second distance L2 from the distal end of the rod 2 positioned at the first position P1 to the distal end 3a of the cap 3 can be longer than the first distance L1 from the distal end of the rod 2 positioned at the first position P1 to the distal end of the hard part 12. In an example, the relationship between the fourth distance L4, which is the inner diameter of the cap (distance in the radial direction R), and the second distance L2 can be 0<L2/L4≤2 when the distal end 3a of the cap 3 is pressed against a tissue such that the tissue positioned on the outward side of the cap 3 is unlikely to enter the inward side of the cap 3. In an example, the relationship therebetween be 0<L2/L4≤1.

In the cap 3, the distal end part of the cap 3 positioned on the distal end side A1 from the step 31 will be referred to as “a distal end tubular part 35”. Similarly to the distal end tubular part 15 of the first embodiment, the distal end tubular part 35 can surround the hemorrhage site H.

Similarly to the distal end tubular part 15 of the first embodiment, the distal end tubular part 35 can be transparent or semi-transparent and have a transparency sufficient to allow the rod 2 to be visually recognized from the outward side of the cap 3. Thus, a practitioner can check the position of the rod 2 from an image captured by the image capturing part 203 of the endoscope 200.

In an example, the distal end tubular part 35 can be formed using a material having an excellent heat resistance. For example, the cap 3 can be formed to include at least one of a ceramic, zirconia, PTFE, and PEEK.

In the distal end tubular part 35, similarly to the distal end tubular part 15A shown in FIG. 11, the inner diameter of an opening 35a (opening diameter) at the distal end can be smaller than the outer diameter of the hard part 12. In addition, the inner circumferential surface of the distal end tubular part 35 can be formed to have a tapered shape such that the inner diameter decreases toward the distal end of the distal end tubular part 35.

According to the treatment tool 100C of the present embodiment, a non-contact state can be maintained in which the distal end (flange 21) of the rod 2 can be kept away from the hemorrhage site H in the tissue of the living body at an appropriate distance. In addition, the hemorrhage site H can be surrounded by the distal end tubular part 35. For this reason, the treatment tool 100C can discharge electricity within an intended range with respect to the hemorrhage site H.

Hereinabove, the third embodiment of the present disclosure has been described in detail with reference to the drawings, but specific constitutions are not limited to this embodiment, and design change and the like within a range not departing from the scope of the present disclosure are also included therein. In addition, the constituent elements shown in the embodiment and the modification example described above can be constituted in suitable combinations.

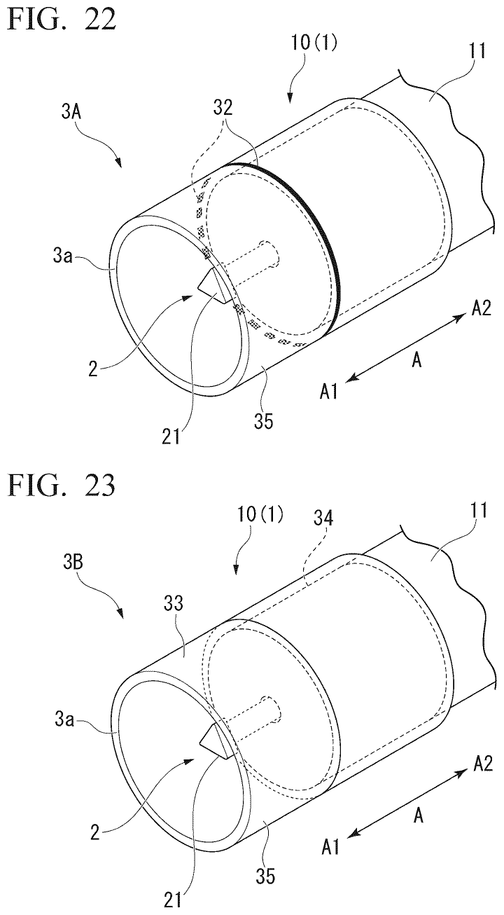

FIG. 22 is an example of a perspective view showing a cap 3A which is a modification example of the cap 3. The cap 3A can have or include a marking 32 in place of the step 31. The marking 32 can be formed in the circumferential direction on the inner circumferential surface of the cap. When the cap 3A is mounted at the distal end of the sheath 1, a practitioner can move the cap 3A until the marking 32 is positioned at the distal end 1a of the sheath 1. Accordingly, the cap 3A can be positionally set with respect to the sheath 1.

FIG. 23 is an example of a perspective view showing a cap 3B which is a modification example of the cap 3. In such an example, the cap 3B does not have the step 31. The cap 3B can have or include a distal end hard part 33 provided on the distal end side A1, and a proximal end soft part 34 provided on the proximal end side A2. The proximal end soft part 34 can be a portion which is softer than the distal end hard part 33 and is likely to be mounted at the distal end of the sheath 1. When the cap 3B is mounted at the distal end of the sheath 1, a practitioner can mount only the proximal end soft part 34 at the distal end 1a of the sheath 1. Accordingly, the cap 3B can be positionally set with respect to the sheath 1.

### Fourth Embodiment

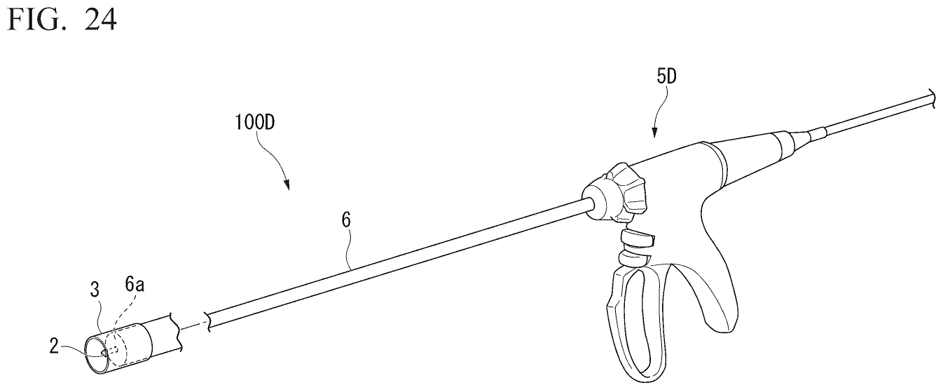

A treatment tool 100D according to a fourth embodiment of the present disclosure will be described with reference to FIG. 24. In the following description, the same reference signs are applied to constituents common to those which have already been described, and duplicate description will be omitted.

FIG. 24 is an example of a perspective view showing the treatment tool 100D. The treatment tool 100D (treatment tool for an endoscope) can be a surgical treatment tool such as a tube for water supply, suction, or hemostasis and can perform incision and coagulation hemostasis. The treatment tool 100D can include a shaft 6, the rod 2, the cap 3 (sheath tube), and a manipulation part 5D.

The shaft 6 can be a rod-shaped member formed using a metal, for example. The rod 2 can be inserted through the shaft 6. The rod 2 can project and retract from a distal end 6a of the shaft 6.

The cap 3 (sheath tube) can be a tubular member which can be attachable and/or detachable with respect to the distal end 6a of the shaft 6 and can have or include electrical insulation properties. The cap 3 can be positionally set with respect to the shaft 6 by a method similar to that of the third embodiment.

Similarly to the treatment tool 100C of the third embodiment, a practitioner can control hemorrhage in the hemorrhage site H using the treatment tool 100D.

According to the treatment tool 100D of the present embodiment, a non-contact state can be maintained in which the distal end (flange 21) of the rod 2 can be kept away from the hemorrhage site H in the tissue of the living body at an appropriate distance. In addition, the hemorrhage site H can be surrounded by the distal end tubular part 35. For this reason, the treatment tool 100D can discharge electricity within an intended range with respect to the hemorrhage site H.

Hereinabove, the fourth embodiment of the present disclosure has been described in detail with reference to the drawings, but specific constitutions are not limited to this embodiment, and design change and the like within a range not departing from the scope of the present disclosure are also included therein. In addition, the constituent elements shown in the embodiment and the modification example described above can be constituted in suitable combinations. The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A treatment tool comprising:
a sheath;
a distal tube that is insulative and extending from a distal end of the sheath;
a wire that is conductive and located inside the sheath;
an electrode inserted in the distal tube and connected to a distal end of the wire; and
wherein the electrode is configured to move between a first position where a distal end of the electrode is located inside the distal tube and a second position where the distal end of the electrode is located distally relative to a distal end of the distal tube, and wherein, when the electrode is located at the first position, the distal end of the distal tube is configured to press a target tissue in a body and the electrode is configured to discharge electricity while maintaining a distance from the target tissue to the electrode.

2. The treatment tool according to claim 1, wherein the distal tube includes a distal tip, and wherein, when the distal end of the electrode is located at the first position, a second distance from the distal end of the electrode to the distal end of the distal tube is longer than a first distance from the distal end of the electrode to the distal end of the distal tip.

3. The treatment tool according to claim 1, wherein a second distance from the distal end of the electrode positioned at the first position to the distal end of the distal tube is substantially equivalent to a first distance from the distal end of the electrode positioned at the second position to the distal end of the distal tube in a longitudinal direction of the sheath.

4. The treatment tool according to claim 1, wherein the distal tube includes a distal tip, the distal tip has a through hole, wherein the electrode has a flange with a larger width perpendicular to a longitudinal axis of the sheath than a main body of the electrode, the flange is located at the distal end of the electrode and a part of the main body located into the through hole when the distal end of the electrode is located at the first position.

5. The treatment tool according to claim 4, wherein, when the distal end of the electrode is located at the first position, the flange abuts the distal tip.

6. The treatment tool according to claim 4, wherein the electrode has a stopper having a larger width perpendicular to a longitudinal axis than the main body of the electrode, the stopper is located proximally relative to the distal tip, and wherein, when the electrode is located at the second position, the stopper is in contact with the distal tip.

7. The treatment tool according to claim 1, wherein the distal tube has a transparency sufficient to allow the electrode to be visually recognized from an outward side of the sheath.

8. The treatment tool according to claim 1, wherein the distal tube has an inner circumferential surface with an inner diameter decreasing from a proximal end of the distal tube toward a distal end of the distal tube.

9. The treatment tool according to claim 1, wherein the distal tube has an extension and contraction part configured to extend and contract.

10. The treatment tool according to claim 1, wherein the distal tube includes a distal tip, and a part of the electrode passes through the distal tip, and wherein in a state in which the distal end of the electrode is adjacent to the distal end of the distal tip, the distal tube is configured to cover the electrode and an area around the tissue.

11. The treatment tool according to claim 1, wherein the electrode includes a main body part and a flange located in a distal part of the main body part, and wherein the flange has a polygonal shape with corners, and the corners are located on a virtual circle concentric with the main body part.

12. The treatment tool according to claim 1, wherein the distal tube includes a distal tip, and wherein an inner diameter of an opening at a distal end of the distal tube is smaller than an outer diameter of the distal tip.

13. The treatment tool according to claim 1, wherein the distal tube includes a distal tip, and a part of the electrode passes through the distal tip, and wherein the sheath includes a tube with the wire inserted therein, and the distal tube is attached to a distal part of the tube, and wherein an outer circumferential surface of the distal tube is positioned outwardly in a radial direction from a connection position of the tube and the distal tip.

14. The treatment tool according to claim 13, wherein the distal tube is a material different from the tube, and a material of the distal tube has a higher heat resistance than a material of the tube.

15. The treatment tool according to claim 1, wherein there is an opening only at the distal end of the distal tube.

16. The treatment tool according to claim 1, wherein the electrode is configured to perform incision treatment when the distal end of the electrode is located at the second position and hemostasis treatment when the distal end of the electrode is located at the first position.

17. The treatment tool according to claim 1, wherein the distal tube surrounds a hemorrhage site in the tissue when the distal end of the distal tube is pressed against the tissue in a longitudinal direction, and electricity is discharged within a discharge space defined by the distal tube.

18. The treatment tool according to claim 1, wherein a material of the distal tube comprises at least one of: ceramic, zirconia, PTFE, or PEEK.

19. The treatment tool according to claim 1, wherein the distal tube includes a distal tip attached to a distal part of the sheath.

20. The treatment tool according to claim 19, wherein the distal tube is a separate component from the distal tip.
